(19) United States
(12) Patent Application Publication
(10) Pub. No.: US 2014/0002792 A1
(43) Pub. Date: Jan. 2, 2014

(54) HOUSING FOR SECURING AND OPTICALLY ALIGNING A CAMERA TO A SCOPE, A METHOD OF ATTACHING SAID HOUSING, AND A KIT INCLUDING SAID MEMBERS

(71) Applicant: Paul A. Filar, Sturgeon Bay, WI (US)

(72) Inventor: Paul A. Filar, Sturgeon Bay, WI (US)

(21) Appl. No.: 13/608,236

(22) Filed: Sep. 10, 2012

Related U.S. Application Data
(63) Continuation-in-part of application No. 12/973,522, filed on Dec. 20, 2010, now Pat. No. 8,262,221.
Application No. 12/973,522 is a continuation-in-part of application No. 12/386,912, filed on Apr. 24, 2009, now Pat. No. 7,883,210.
Application No. 12/386,912 is a continuation-in-part of application No. 29/422,585, filed on May 22, 2012, now abandoned.

(51) Int. Cl. G03B 17/02; A61B 3/14

(52) U.S. Cl. CPC G03B 17/02; A61B 3/14
USPC 351/206; 396/535

(57) ABSTRACT

A housing is disclosed for optically aligning and securing a camera to an aperture formed through a tubular barrel of a scope. The housing has a first attachment portion sized and configured to be removably secured to at least a portion of the tubular barrel and a second attachment portion having a first surface, a second surface and an aperture formed therethrough. The first surface is secured to the first attachment portion and the second surface is sized and configured to removably retain a camera, such that the camera is in optical alignment with both the aperture formed through the second attachment portion and the aperture formed through the tubular barrel of the scope. A combination housing and camera, a method of attaching the housing to the scope, and a kit containing the necessary items is also disclosed.

### RELATED U.S. APPLICATION DATA

This application is a Continuation-In-Part of application Ser. No. 12/973,522, filed on Dec. 20, 2011, which in turn is a Continuation-In-Part of application Ser. No. 12/386,912, filed Apr. 24, 2009, now U.S. Pat. No. 7,883,210, and of Design Application No. 29/422,585 filed on May 22, 2012.

### FIELD OF THE INVENTION

This invention relates to a housing for securing and optically aligning the shutter and lens of a camera to an aperture formed through a tubular barrel of a scope, a method of attaching the housing to the tubular barrel, and a kit including the required members. More specifically, this invention relates to a removable housing which can secure and optically align a camera to an aperture formed through a tubular barrel of a portable opthalmoscope.

### BACKGROUND OF THE INVENTION

Scopes of various structures and functions, including but not limited to: opthalmoscopes, periscopes, telescopes, microscopes, etc. can be used as viewing and/or magnification instruments. An opthalmoscope is an instrument for examining the interior structures of the eye, especially the retina. An opthalmoscope consists of a mirror that reflects light into the eye and a central hole through which the eye is examined. A periscope is a tubular optical instrument that contains reflecting elements, such as mirrors and prisms to permit observation from a position displaced from a direct line of sight. A telescope is an instrument of various arrangement of lenses or mirrors or both that gathers visible light, permitting observation of distant objects. A microscope is an optical instrument that uses a lens or a combination of lenses to produce magnified images of objects too small to be seen by the unaided eye.

A camera is an apparatus for taking photographs or visual images and consists of a lightproof enclosure having a shutter and lens through which the image of an object is recorded. The photograph, picture or visual image can be stored and/or displayed electronically, or be recorded on a photosensitive film or plate. Today, many cameras are small and portable and some have electronic and/or digital capabilities which allow the images to be immediately viewed and/or be stored electronically. Many cameras include a monitor for directly viewing the photos, pictures or visual images and also contain at least one port so that the camera can be connected to another device, such as a computer, a laptop, an electronic notebook, a monitor, a screen, a television set, an iPad, an iPhone, a smart phone, a cellular phone, etc. so that the photos, pictures or visual images can be displayed. Today, many cellular phones, smart phones, computer devices and other electronic devices include built-in camera or have the ability of having a camera externally attached to them. Examples of devices which include a camera include: iPhones, iPads, smart phones, most cellular phones, video devices, computers, laptops, electronic notebooks, etc. The camera may be designed to take still pictures, videos, or a combination of still pictures and videos.

It should be understood that in our rapidly changing era, images can be electronically recorded, stored, transmitted and/or viewed in a variety of ways. Wireless storage and/or transmission from a camera to another location or device, such as to an electronic health record site, to cloud computing, to wireless monitors, etc. can all be utilized.

In the field of Optometry and Ophthalmology (human and animal) it has been found by the present inventor that by using a housing to optically align and secure the shutter and lens of a camera to an aperture formed through a tubular barrel of an opthalmoscope, that one can photograph and/or take videos of the anterior segments and retina of the eye. Such a housing allows a Doctor of Optometry, an MD, a researcher or some other person to easily observe various diseases, conditions and trauma in patients, such as cancer, diabetes, high blood pressure, glaucoma, STD's, anemia, MS, high cholesterol, lupus, thyroid, sleep apnea, shingles, foreign objects, hypertension, diabetic retinopathy and papilledema. In addition, the housing can be used to secure and optically align the shutter and lens of a camera or a device which includes a camera, such as an iPhone, an iPad, a smart phone, a cell phone, a video device, etc. to various other kinds of scopes. It is foreseen that the housing could be used to secure and optically align the shutter and lens of a camera or video device to a periscope, a telescope, a microscope, a stereoscope, a monocular, binoculars, or to any other instrument that uses electronics or other processes to magnify or view an object. A stereoscope is an instrument with two eyepieces used to impart a three-dimensional effect to two photographs of the same scene from slightly different angles. A monocular is an instrument relating to or intended to be used by only one eye. Binoculars are an instrument involving both eyes at the same time and functions to produce stereoscopic vision.

Currently, there are many commercial, industrial, defense and governmental applications where a housing is needed which can secure and optically align the shutter and lens of a camera to an aperture formed through a tubular barrel of a scope.

### SUMMARY OF THE INVENTION

Briefly, this invention relates to a housing for optically aligning and securing a camera to an aperture formed through a tubular barrel of a scope. The housing has a first attachment portion sized and configured to be removeably secured to at least a portion of the tubular barrel and a second attachment portion having a first surface, a second surface and an aperture formed therethrough. The first surface is secured to the first attachment portion and the second surface is sized and configured to removably retain a camera, such that the camera is in optical alignment with both the aperture formed through the second attachment portion and the aperture formed through the tubular barrel of the scope.

This invention also relates to a combination including a camera and a housing. The housing permits the camera to be optically aligned with an aperture formed through a tubular barrel of a scope. The housing has a first attachment portion sized and configured to be removeably secured to at least one end of the tubular barrel and a second attachment portion having a first surface, a second surface and an aperture formed therethrough. The first surface is secured to the first attachment portion and the second surface is sized and configured to removably retain the camera, such that the camera is in optical alignment with both the aperture formed through the second attachment portion and the aperture formed through the tubular barrel of the scope.

This invention further relates to a method for optically aligning and securing a camera to a tubular barrel of a scope wherein the tubular barrel has an aperture formed therethrough. The method includes the steps of forming a housing having a first attachment portion sized and configured to be removeably secured to at least one end of the tubular barrel and a second attachment portion having a first surface, a second surface, and an aperture formed therethrough. The first surface is secured to the first attachment portion and the second surface sized and configured to removably retain the camera. The method also includes positioning and securing the first attachment portion onto one end of the tubular barrel and then inserting the camera into the second attachment portion such that the camera is in optical alignment with both the aperture formed through the second attachment portion and the aperture formed through the tubular barrel.

Still further, this invention relates to a kit which includes a case having a first portion joined to a second portion. The first and second portions are movable between an open position, wherein the case is open, and a dosed position, wherein the case is dosed. The case has a locking mechanism capable of securing the first portion to the second portion when the first and second portions are in the closed position. The case also includes at least one foam member positioned within at least one of the first and second portions. The foam member has at least three separate and distinct cutouts formed therein. Each of the cutouts is sized and configured to snuggly engage and temporarily hold an object inserted therein. The kit also includes an opthalmoscope removably positioned in one of the cutouts. The opthalmoscope having a tubular barrel with an aperture formed therethrough. The kit also includes a camera removably positioned in one of the cutouts. The camera has a button which can be activated to take a picture. The kit further includes a housing removably positioned in one of the cutouts. The housing is capable of optically aligning and securing the camera to the aperture formed through the opthalmoscope. The housing has a first attachment portion sized and configured to be removeably secured to at least one end of the opthalmoscope and a second attachment portion having a first surface, a second surface, and an aperture formed therethrough. The first surface is secured to the first attachment portion and the second surface is sized and configured to retain the camera, such that the camera is in optical alignment with both the aperture formed through the second attachment portion and the aperture formed through the tubular barrel.

The general object of this invention is to provide a housing for securing and optically aligning the shutter and lens of a camera to an aperture formed through a tubular barrel of a scope, a method of attaching said housing to the tubular barrel of a scope, and a kit including the required members. A more specific object of this invention is to provide a housing which can secure and optically align the shutter and lens of a camera to an aperture formed through a tubular barrel of a portable opthalmoscope.

Another object of this invention is to provide a combination camera and housing which will allow the camera to be secured to and optically aligned with an aperture formed through a tubular barrel of a scope.

Another object of this invention is to provide a method of removably securing a camera to a housing and then removably securing the housing onto a tubular barrel of a scope.

A further object of this invention is to provide a method of removably securing a housing onto a tubular barrel of a scope and then removably securing a camera to the housing.

Still another object of this invention is to provide a kit which includes a case retaining an opthalmoscope, a camera, and a housing for securing and optically aligning the shutter and lens of the camera to an aperture formed through a tubular barrel of an opthalmoscope.

Still further, an object of this invention is to provide a housing which can permanently retain a camera and optically align the shutter and lens of the camera to an aperture formed through a tubular barrel of an opthalmoscope.

Other objects and advantages of the present invention will become more apparent to those skilled in the art in view of the following description and the accompanying drawings.

### BRIEF DESCRIPTION OF THE DRAWINGS

FIG. 1 is a side view of a housing which is capable of securing and optically aligning the shutter and lens of a camera to an aperture formed through a tubular barrel of a scope.

FIG. 2 is a perspective view of a camera having a shutter and lens.

FIG. 3 is a side perspective view of a scope.

FIG. 4 is an end view of a portion of the scope shown in FIG. 3 taken along line 4-4.

FIG. 5 is a right end view of the housing shown in FIG. 1.

FIG. 6 is a left end view of the housing shown in FIG. 1.

FIG. 7 is a bottom view of the housing shown in FIG. 1.

FIG. 8 is an alternative embodiment of the housing shown in FIG. 6 wherein the upper surface has a single cantilevered member which can be depressed to activate the camera.

FIG. 9 is a side view of another embodiment of a housing which is capable of securing and optically aligning the shutter and lens of a camera to an aperture formed through a tubular barrel of a scope wherein the camera has a built-in monitor.

FIG. 10 is a right end view of the housing shown in FIG. 9.

FIG. 11 is a left end view of the housing shown in FIG. 9.

FIG. 12 is a bottom view of the housing shown in FIG. 9.

FIG. 13 is a side view of a third embodiment of a housing which is capable of securing and optically aligning the shutter and lens of a camera to an aperture formed through a tubular barrel of a scope wherein an external device which has a built-in camera can inserted and then removed from the housing.

FIG. 14 is a right end view of the housing shown in FIG. 13.

FIG. 15 is a left end view of the housing shown in FIG. 13.

FIG. 16 is a side view of the housing shown in FIG. 13 depicting the second attachment member having been rotated 90 degrees relative to the first attachment portion.

FIG. 17 is a front view of a cellular phone having a built-in camera.

FIG. 18 is a right side view of the cellular phone shown in FIG. 17 depicting an on/off button and an activation button for the camera.

FIG. 19 is a left side view of the cellular phone shown in FIG. 17 showing a port.

FIG. 20 is a back view of the cellular phone shown in FIG. 17.

FIG. 21 is a bottom view of the cellular phone shown in FIG. 17 depicting a removable cover enclosing another port.

FIG. 22 is a side view of the cellular phone shown in FIG. 17 being removably attached to the housing shown in FIG. 13.

FIG. 23 is a front view of a fourth embodiment of a housing which is capable of securing and optically aligning the shutter and lens of a camera to an aperture formed through a tubular barrel of a scope wherein the housing can temporarily secure a cellular phone having a built-in camera.

FIG. 24 is a right end view of the housing shown in FIG. 23.

FIG. 25 is a left end view of the housing shown in FIG. 23.

FIG. 26 is a back view of the housing shown in FIG. 23.

FIG. 27 is a perspective view of an open case wherein the case includes a foam member with a plurality of cutouts for snuggly holding various objects and devices.

FIG. 28 is a perspective view of a dosed case.

### DETAILED DESCRIPTION OF THE INVENTION

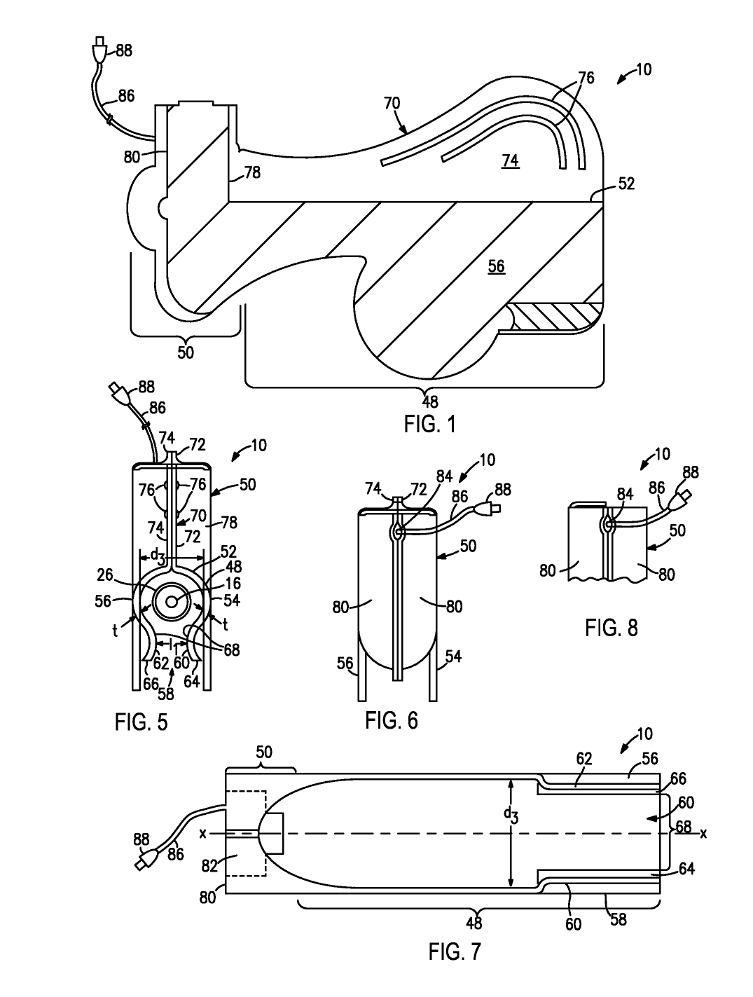

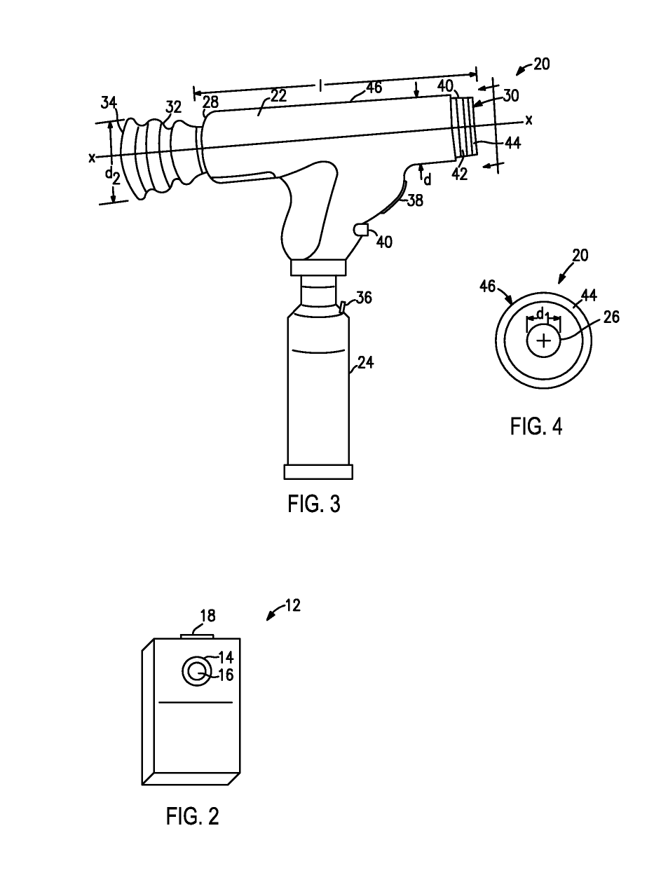

Referring to FIGS. 1-4, a housing 10 is shown in FIG. 1 which is capable of securing and optically aligning a camera and/or video device 12, shown in FIG. 2, to an aperture 26 formed through a tubular barrel 22 of a scope 20, see FIGS. 3-4. The housing 10 can be an integral member or be formed from two or more members that are joined together. The housing 10 can be formed in a variety of ways. For example, the housing 10 can be injection molded. The camera and/or video device 12 can be a camera, a video device or a combination of both. A camera is an apparatus capable of taking photos, photographs or pictures and is sometimes referred to as taking still pictures or visual images. A video device is an apparatus capable of recording events over a period of time. The video camera normally records images on videocassettes, videotapes, memory cards, etc. for playback on a screen, monitor, television set or some other device. A combination camera and video device can take both still pictures and well as videos. To assist the reader in understanding this invention, the word “camera” will be used to refer to an apparatus which can be a camera with or without video capabilities.

The camera 12 can vary in size, shape, construction and price. Today, there are many manufacturers who commercially sell a wide variety of cameras. The camera 12 can be portable. Many cameras 12 are rather compact and some can fit into the pocket of a pair of pants or into a handbag or purse. The camera 12 can be an electronic device. The camera 12 can operate using analog or digital technology. Alternatively, the camera 12 can use photosensitive film or plate to record and store images. Today, most cameras 12 record and store images in electronic form. The camera 12 could also be a sophisticated model with special filters and/or electronic capabilities. For example, the camera 12 could include 3D imaging, Fundus Autofluorescence (FAF), etc. The camera 12 can also be incorporated into various other electronic devices, such as into cellular phones, smart phones, computers, laptops, electronic notebooks, iPads, and/or other electronic gadgets. The iPad, iPhone, smart phones, laptops, electronic notebooks, etc. are a few examples of devices that have a built-in camera. Alternatively, the camera 12 can be externally attached to some of electronic device, such as to a computer.

Referring to FIG. 2, the camera 12 has a shutter 14 movably mounted over a lens 16. The camera 12 also has an activation mechanism 18, such as a button, connected to the shutter 14 which can be depressed to activate the shutter 14 and take a photograph, picture or visual image. The internal components of a camera 12 are well known to those skilled in the camera art. Today many cameras 12 contain electronics which allow the photos, photographs, pictures or images to be recorded, stored and then transferred to another device, such as a computer, a monitor, a television screen or some other type of image viewing device where the photos, photographs, pictures or images can be visually shown and displayed. Alternatively, the camera 12 can include a monitor which allows the photos, photographs, pictures or images to be immediately viewed. The electronics within the camera 12 allow the photos, photographs, pictures or images to be viewed on the monitor. The size of the monitors can vary.

It should be understood that the terms photos, photographs, pictures and images are being used interchangeably. The images taken by the camera 12 can be electronic images or be images which have been transferred to a photosensitive film or plate. Many cameras 12 are small and compact units which have electronic, analog or digital capabilities, that allow the images to be electronically recorded, stored and viewed either on a built-in monitor or on a distal monitor. The camera 12 usually has at least one port which permits the camera 12 to be electronically connected by a wire to another device, such as a computer, a laptop, an electronic notebook, a monitor, a screen, a television set, an iPad, an iPhone, a smart phone, a cellular phone, etc. so that the photos, photographs, pictures or images can be displayed. Today, many cellular phones, computer devices and other electronic devices include a built-in camera.

Referring to FIG. 3, the scope 20 can be almost any kind of scope now in existence. For example, the scope 20 can be an opthalmoscope, a periscope, a telescope, a microscope, a stereoscope, or any other instrument that uses electronics or other processes to magnify or view an object. The scope 20 can also be in the form of a monocular or binoculars. The scope 20 can be a portable scope that can be hand held. The scope 20 is depicted as an opthalmoscope used by a Doctor of Optometry to view the anterior segments and retina of the eye. The scope 20 has a tubular barrel 22 with a longitudinal central axis X-X. The tubular barrel 22 can be formed from a variety of materials. The tubular barrel 22 has a length l of about 3 to about 6 inches. Desirably, the length l of the tubular barrel 22 is about 5 inches in length. The tubular barrel 22 has an outside diameter d which can be constant throughout its length l or which can vary over its length l. Desirably, the outside diameter d will remain approximately constant throughout its length l. The outside diameter d should be greater than about 0.5 inches. Desirably, the outside diameter d ranges from between about 1 inch to about 2 inches. More desirably, the outside diameter d is about 1.25 inches.

The tubular barrel 22 is attached to a handle 24. The handle 24 can be permanently secured to the tubular barrel 22 or can be removably attached to the tubular barrel 22. The handle 24 should be attached to the tubular barrel 22 somewhere along its length l. The handle 24 allows a doctor to comfortably hold and position the scope 20 in one hand. The doctor can then move and position the scope 20 against the patients head or face, approximate one eye, and move the scope to the other eye when needed. The handle 24 can be aligned at an angle to the tubular barrel 22. For example, the handle 24 can be aligned at about a 90 degree angle to the tubular barrel 22. Alternatively, the handle 24 can be aligned at an angle which ranges from between about 60 degrees to about 120 degrees relative to the tubular barrel 22. Desirably, the handle 24 is aligned at an angle which ranges from between about 75 degrees to about 105 degrees to the tubular barrel 22. The handle 24 can be formed from a variety of materials. The handle 24 can contain a power unit (not shown), such as a battery.

Referring to FIGS. 3 and 4, the tubular barrel 22 has an aperture 26 is formed through its length l. Desirably, the aperture 26 is coaxially aligned with the longitudinal central axis X-X of the tubular barrel 22. The aperture has a diameter d₁. The diameter d₁ of the aperture 22 can vary. Desirably, the diameter d₁ of the aperture 22 is less than about 0.5 inches. More desirably, the diameter d₁ of the aperture 22 is less than about 0.4 inches. Even more desirably, the diameter d₁ of the aperture 22 is less than 0.25 inches.

The scope 20 has a first end 28 and a spaced apart second end 30. Removably attached to the first end 28 is a first member 32. The first member 32 can vary in configuration but desirably is a generally hollow, tube shaped profile. The first member 32 can be formed from any soft material. Rubber is a desirable material from which to form the first member 32. The first member 32 has a distal end 34 which is designed to be placed in direct contact with a patient's head or face, such that it surrounds the eye. Desirably, the first member 32 has a conical shape which tapers outwardly toward the distal end 34. The first member 32 has an outside diameter d₂. The maximum outside diameter d₂ occurs approximate the distal end 34. The first member 32 can contain two or more bellows along its length which allow the first member 32 to contract or to acquire a shorter length as the scope 20 is brought into contact and pressed against a patients head or face. In other words, the first member 32 acts like a shock absorber as the scope 20 is moved and positioned about a patient's eye. The maximum outside diameter d₂ of the first member 32 can vary. Desirably, the maximum outside diameter d₂ of the first member 32 can range from about 1.5 inches to about 2.5 inches. More desirably, the maximum outside diameter d₂ of the first member 32 can range from about 1.5 inches to about 2 inches. Even more desirably, the maximum outside diameter d₂ of the first member 32 will be about 1.75 inches.

Still referring to FIG. 3, the second end 30 of the scope 20 is directly aligned with the first end 28. The second end 30 is the end of the scope 20 through which the doctor normally looks to view the anterior segments and retina of a patient's eye. The scope 20 also has one or more buttons or control knobs. In FIG. 3, the scope 20 is shown having an on/off button 36 which can be pressed to turn the scope 20 on and which can be pressed again to turn the scope 20 off. The scope 20 also has two control knobs 38 and 40. The control knob 38 can be adjusted to control the magnification of the scope 20 while the control knob 40 can be adjusted to change the aperture 26. Additional buttons and control knobs may be present, if needed. The scope 20, shown in FIG. 3, is an opthalmoscope, commercially available from Welch Allyn Inc. having an office at 4341 State Street, Skaneateles, N.Y., 13153-0020 USA.

Removably attached to the second end 30 of the scope 20 is a second member 42. The second member 42 can vary in configuration but desirably has a generally hollow, tube shaped profile. The second member 42 can be formed from any soft material. Rubber is a desirable material from which to form the second member 42. The second member 42 has a distal end 44 which is designed to be placed in direct contact or close to the doctor's dominate eye. The doctor will peer through the aperture 26, formed through the tubular barrel 22 of the scope 20, via the second member 42.

Still referring to FIG. 3, the tubular barrel 22 has an outer surface 46. The outer surface 46 can be formed from a variety of materials, including but not limited to: plastic, metal, a metal alloy, aluminum, titanium, a composite material, etc.

It should be noted that the general size, shape and configuration of the scope 20 can vary depending on what type of scope it is.

Referring again to FIG. 1, the housing 10 is designed to be removably secured or mounted onto the outer surface 46 of the tubular barrel 22 of the scope 20. By “removably secured or mounted” it is meant that the housing 10 can be secured to the outer surface 46 of the tubular barrel 22 and can be removed from the tubular barrel 22 multiple times, in an easy and quick manner. The housing 10 is not designed to be permanently secured to the scope 20 but could be so designed, if desired. The housing 10 is designed to secure the camera 12 and optically align the shutter 14 and lens 16 of the camera 12 with the aperture 26 formed through the tubular barrel 22 of the scope 20. This arrangement permits the shutter 14 and the lens 16 to be coaxially aligned with the aperture 26 along the longitudinal central axis X-X. This arrangement also permits a visual image to be taken of the patient's eye when the activation mechanism 18 on the camera 12 is pressed by the doctor.

In FIG. 1, the housing is shown 180 degrees from its orientation as it would appear when positioned on the scope 20, depicted in FIG. 3.

Referring now to FIGS. 1 and 5-8, the housing 10 has a first attachment portion 48 and a second attachment portion 50. The first attachment portion 48 is sized and configured to be removably secured to at least a portion of the scope 20. Desirably, the first attachment portion 48 will be removably secured to at least an end of the scope 20. More desirably, the first attachment portion 48 will enclose the second member 42 and distal end 44 of the scope 20. Even more desirably, the first attachment portion 48 will cover a portion of the outer surface 46 of the tubular barrel 22 and cover the second end of the scope 20. The housing 10 can be configured to mount onto or over a portion of the outer surface 46 of the tubular barrel 22 or onto the second member 42 so that the second end 30 of the scope 20 is surrounded and covered by the housing 10. Desirably, the housing 10 will cover a portion of the outer surface 46 of the tubular barrel 22 as well as the second end 30.

The first attachment portion 48 can vary in configuration. As depicted in FIG. 5, the first attachment portion 48 has a generally C-shaped cross-sectional profile spanning an arc of at least about 270 degrees. The first attachment portion 48 has a top wall 52 and a pair of flexible sidewalls 54 and 56 extending outward and downward from said top wall 52. The top wall 52 and the pair of flexible sidewalls 54 and 56 create the generally C-shaped profile. Each of the pair of sidewalls 54 and 56 can extend downward from between about 1 to 3 inches from the top wall 52. Desirably, each of the pair of sidewalls 54 and 56 will extend downward from between about 1.5 inches to about 2.5 inches from the top wall 52. Even more desirably, each of the pair of sidewalls 54 and 56 will extend downward about 2 inches from the top wall 52. By sizing the pair of sidewalls 54 and 56 to extend downward from the top wall 52 by such a distance, one can be assured that the housing 10 will be secured to the scope 20. In addition, the length of the pair of sidewalls 54 and 56 will contact the sides of the scope that extend downward from the tubular barrel 22 and prevent the housing 10 from rotating on the scope 20. This is important because the housing 10 must be secure and stationary when it is mounted on the scope 20.

Referring to FIG. 5, it should be noted that the pair of sidewall 54 and 56 can be identical in size and length or they can be different in either size or length, or both. Desirably, the pair of sidewalls 54 and 56 is identical to one another. Each of the pair of sidewalls 54 and 56 can vary in thickness t. Desirably, each of the pair of sidewalls 54 and 56 has the same thickness t. The thickness t of each of the pair of sidewalls 54 and 56 can range from between about 0.01 inches to about 0.3 inches. Desirably, the thickness t of each of the pair of sidewalls 54 and 56 will be from about 0.05 inches to about 0.1 inches. Each of the pair of sidewalls 54 and 56 is a flexible member which is capable of flexing horizontally outward from an initial orientation. Each of the pair of sidewalls 54 and 56 should be able to flex or move outwardly from an initial orientation by at least about 0.3 inches. Desirably, each of the pair of sidewalls 54 and 56 should be able to flex or move outwardly from an initial orientation by at least about 0.5 inches. More desirably, each of the pair of sidewalls 54 and 56 should be able to flex or move outwardly from an initial orientation by at least about 1 inch. It should be noted that the material from which the housing 10 is constructed, the thickness t of each of the pair of sidewalls 54 and 56, the particular configuration of the housing 10, and the way the pair of sidewalls 54 and 56 are formed relative to the top wall 52 will all dictate how much each of the pair of sidewalls 54 and 56 can flex or move outward from an initial orientation.

Each of the pair of sidewalls 54 and 56 also has a resiliency to return to its initial or original position or orientation after the force required to make each of them flex or move outward has been removed. This resiliency can be created at least partially by the structural shape of each of the pair of sidewalls 54 and 56, at least partially by the material from which each of the pair of sidewalls 54 and 56 is formed, or from the structural characteristics of each of the pair of sidewalls 54 and 56 themselves. Those skilled in the art will know various ways in which resiliency can be created or enhanced in a given member.

Still referring to FIG. 5, the C-shaped profile has an opening 58 formed therein. The opening 58 creates an open bottom on the C-shaped profile. The opening 58 can span a horizontal distance of from about 0.75 to about 1.5 inches. Desirably, the opening 58 will span a horizontal distance of about 1.25 inches. The C-shaped profile has an internal diameter d₃ which is slightly larger than said outside diameter d of the tubular member 22. The internal diameter d₃ of the C-shaped profile should be at least about 1 inch. Desirably, the internal diameter d₃ of the C-shaped profile is at least about 1.25 inches. The opening 58 provides the mechanism for securing the housing 10 to tubular barrel 22 of the scope 20. As the housing 10 is pressed downward against the outer surface 46 of the tubular barrel 22, the pair of sidewalls 54 and 56 will flex outwardly thereby enabling the internal diameter d₃ of the housing 10 to encircle a portion of the outside diameter d of the tubular barrel 22.

Returning to FIGS. 5 and 7, one will clearly see that a foot 60 is formed on a portion of the sidewall 54 and a foot 62 is formed on a portion of the sidewall 56. The feet 60 and 62 can vary in configuration. Desirably, each of the feet 60 and 62 has an arcuate profile with a distal end 64 and 66, respectively. The distal ends 64 and 66 can be flat and be aligned perpendicular or at an angle to the pair of sidewalls 54 and 56. The feet 60 and 62 can be configured such that as the housing 10 is pressed downward into contact with the outer surface 46 of the tubular barrel 22, the ends 64 and 66 of each of the feet 60 and 62 will cause the pair of sidewalls 54 and 56 to flex and move horizontally outward. By forming the ends 64 and 66 at an angle or taper, one can decrease the force needed to press the housing 10 onto the outer surface 46 of the tubular barrel 22. This will allow the housing 10 to be mounted on or secured to the tubular barrel 22 of the scope 20 in an easy and reliable fashion.

It should be understood that the feet 60 and 62 and their uniquely shaped ends 64 and 66 will facilitate mounting of the housing 10 onto the tubular barrel 22. The exact shape of the feet 60 and 62 will definitely decrease the amount of force needed to mount or press the housing 10 onto the tubular barrel 22 of the scope 20.

Returning to FIG. 5, one can clearly see that the length l, located between the pair of feet 60 and 62 will create a neck portion 68 or area between the pair of flexible sidewalls 54 and 56. This neck portion 68 is narrower than the distance between the remainder of the pair of sidewalls 54 and 56. This neck portion 68 will assist in holding the housing 10 secure to the tubular barrel 22 of the scope 20 and prevent the housing 10 from rotating on the tubular barrel 22 once the pair of flexible sidewalls 54 and 56 return to or toward their initial orientation and position.

Referring again to FIGS. 1 and 5, the housing 10 also has a grasping mechanism 70 which is secured to the top wall 52. The grasping mechanism 70 can vary in size, construction and configuration. The grasping mechanism 70 is depicted as an upwardly extending fin. The fin is merely one example of what the grasping mechanism 70 can look like. The grasping mechanism 70 has a pair of sidewalls 72 and 74 which extend upward from the top wall 52. The grasping mechanism 70 can include one or more ribs, embossments or indentations 76. Two ribs 76, 76 are shown in FIG. 1 with each having a curved or flowing profile. The exact number, shape and configuration of the ribs, embossments or indentations can vary. Each of the ribs 76, 76 projects outward from the sidewalls 72 and 74. However, if indentations 76 were used, they would extend into each of the sidewalls 72 and 74. The ribs, embossments or indentations 76 function to provide a non-slip finger griping surface. The ribs, embossments or indentations 76 provide a means for a person, such as the doctor, to grasps the housing 10 and easily apply or remove it from the tubular barrel 22 of the scope 20. The grasping mechanism 70 can be used to both attach the housing 10 to the tubular barrel 22 of the scope 20 and to remove or separate the housing 10 from the tubular barrel 22 of the scope 20. The grasping mechanism 70 facilitates mounting and securing the housing 10 on the tubular barrel 22 of the scope 20 and removing the housing 10 from the tubular barrel 22 of the scope 20. The grasping mechanism 70 is designed to be grabbed in one hand between the thumb on one sidewall 72 and two or more fingers on the opposite sidewall 74. By pushing down or pulling up, one can easily and quickly attach or remove, respectively, the housing 10 from the tubular barrel 22 of the scope 20.

It should be understood that the grasping mechanism 70 can be reconfigured to include a handle, finger and/or thumb openings, and/or an ergonomically shaped palm structure, if desired.

Still referring to FIGS. 1 and 5-8, the second attachment portion 50 of the housing 10 includes a first surface 78 and a second surface 80. The first surface 78 is secured to the first attachment portion 48. The second surface 80 is sized and configured to cooperate with the first surface 78 to at least partially enclose or retain the camera 12. In FIG. 1, the second surface 80 cooperates with the first surface 78 to provide a chamber 82, see FIG. 7, into which the camera 12 can be inserted and held in a stationary position. The chamber 82 can vary in size and geometrical shape. It is important that the camera 12 does not rotate or move its X, Y and Z axes once it is secured to the housing 10. The camera 12 can be permanently secured to the housing 10 or it can be removably secured to the housing 10. By “removably secured’ it is meant that the camera 12 could be inserted into the housing 10 and then be later removed and separated from the housing 10. This removal feature is very desirable because today many cellular phones and smart phones include a built-in camera 12 which could easily be secured to the housing 10 to record and store images taken with the scope 20. The doctor or person using the scope 20 could then remove his or her cellular phone or smart phone and use it to make or receive calls. In addition, many cellular phones today are referred to as “smart phones” because they can accommodate various electronic applications which allow them to provide various functions. For example, a smart phone can include an application that can provide a Global Positioning System (GPS), give driving directions, provide the current time, show a calendar, provide weather forecasts, provide news, etc. Because of these various features, the smart phone owner may want to remove his smart phone from the housing 10 when he or she is not using the scope 20.

Regardless of how the camera 12 is secured to the second attachment portion 50 of the housing 12, it is important that the camera 12 be held secure when it is attached to the housing 10. The reason for this is that the shutter 14 and lens 16 of the camera 12 must be optically aligned with the aperture 26 formed through the tubular barrel 22 of the scope 20. This will insure that a high quality image of the object that the scope 20 is focused on can be taken. In the case of an opthalmoscope, the doctor will want to obtain a high quality image of the anterior segments and/or retina of a patient's eye.

The second attachment portion 50 can have a generally rectangular configuration which is sized and configured to permanently retain a camera 12 therein or to temporarily receive and retain a cellular phone having a camera built therein. Alternatively, the second attachment portion 50 can be a partially enclosed chamber 82 or a fully enclosed chamber 82 with an opening aligned with the shutter 14 and lens 16 of the camera 12.

Referring again to FIGS. 2-4, the tubular barrel 22 of the scope 20 has an outer surface 46 and a longitudinal central axis X-X. The aperture 26 formed through the tubular barrel 22 is coaxially aligned with the longitudinal central axis X-X. The camera 12 has a shutter 14 and a lens 16 which are optically aligned with the aperture 26. In order to insure that the housing 10 will be held secure on the tubular barrel 22, one can vary the coefficient of friction between the outer surface 46 of the tubular barrel 22 and the material forming the internal diameter d₃ of housing 10. This difference in coefficient of friction only has to be present in the C-shaped profile portion of the first attachment portion 48. The outer surface 46 of the tubular barrel 22 can have a coefficient of friction which is greater than or less than the coefficient of friction of the first attachment portion 48. For example, the outer surface 46 of the tubular barrel 22 can have a coefficient of friction which is greater than the coefficient of friction of the first attachment portion 48. Alternatively, the outer surface 46 of the tubular barrel 22 can have a coefficient of friction which is less than the coefficient of friction of the first attachment portion 48.

Referring again to FIGS. 1 and 5-8, the housing 10 further contains an opening 84 formed in the second surface 80 through which an electrical cord 86 to the camera 12 can pass. The size of the opening 84 can vary. Typically, the opening 84 is less than about 0.25 inches in diameter. The electrical cord 86 can contain a Universal Serial Bus (USB) plug 88 on its distal end. The USB plug provides a means for electronically downloading and/or storing the images captured by the camera 12 to an external device such as a computer, an iPad, an iPhone, a monitor, a television set, or some other type of external device which will allow the images to be visually viewed and/or stored. The original USB was introduced in January, 1996 for the purpose of providing data transfer from personal computers to external devices connected to the personal computer.

It should be noted that the opposite end of the cord 86 can be removed from the camera 12 when not needed. In other words, the cord 86 is not permanently attached to the camera 12. The cord 86 can be stored in a safe place until the images recorded by the camera 12 need to be downloaded or transferred to an external device. At that time, one end of the cord 86 can be connected to the camera 12 and the opposite end can be connected to the external device for viewing and/or storing the visual images.

Referring now to FIGS. 9-12, a second embodiment of a housing 10′ is shown. The housing 10′ functions to optically align and secure a camera 12 to an aperture 26 formed through a tubular barrel 22 of the scope 20. The housing 10′ has a first attachment portion 48′ secured to a second attachment portion 50′. The second attachment portion 50′ has a first surface 78′, a second surface 80′ and an aperture 112 formed therethrough. One difference between the housing 10 and the housing 10′ is that the first attachment portion 48′ includes a circular rib, embossment or indentation 77, in addition to the pair of ribs, embossments or indentations 76, 76. The circular rib, embossment or indentation 77 illustrates that different structures can be utilized which can vary in size, shape and geometry. Another difference between the housings 10 and 10′ is that in the housing 10′, the second attachment portion 50′ is permanently secured to the first attachment portion 48′ at the first surface 78′. The second attachment portion 50′ is designed to completely encompass a camera 12. The camera 12 includes a built-in monitor 90 that is visible on one of its major surfaces. The monitor 90 can vary in size and shape. Normally, the larger the dimensions of the monitor 90, the better. The monitor 90 can have a square configuration. Desirably, the monitor 90 has a rectangular configuration. The monitor 90 can be a rectangle having a length l of at least about 1.5 inches and a width w of at least about 1 inch. Desirably, the monitor 90 can have a length l which ranges from between about 1.5 inches to about 5 inches, and a width w which ranges from between about 1 inch to about 4.5 inches. More desirably, the monitor 90 can have a length l which ranges from between about 1.5 inches to about 3 inches, and a width w which ranges from between about 1 inch to about 2.5 inches.

Referring to FIG. 11, the second attachment portion 50′ is configured such that at least about 50% of a major surface of the camera 12 is exposed. The monitor 90 is visible on one of the major surfaces of the camera 12. Desirably, at least about 60% of a major surface of the camera 12 is exposed. More desirably, at least about 75% of a major surface of the camera 12 is exposed. In other words, the monitor 90 is visible on at least about 60% or on at least 75% of a major surface of the camera 12.

Referring again to FIG. 9, the second attachment portion 50′ has an opening 92 formed therein which exposes a portion of the camera 12. The opening 92 coincides with a USB port formed in the camera. As mentioned above with reference to the first embodiment of the housing 10, an electrical cord 86 (not shown) can be connected to the USB port so that images recorded and captured by the camera 12 can be transferred and stored on an external device. The opening 92 also exposes a receptacle 96 in which a memory card 98 can be inserted. A memory card 98 is used to store images recorded by the camera 12. The memory card 98 is designed to be easily removed from the camera 12 and can be directly inserted into an external device, such as a computer, whereby the electronic images can be downloaded and viewed or stored. Those skilled in the art of cameras and computers will be well aware of the different types of memory cards 98 which are commercially available and the capacity of the various models.

Referring now to FIGS. 9, 10 and 12, one can see that the second attachment portion 50′ also has another opening 100. This opening faces towards the first attachment portion 48′ and is sized and shaped to allow an on/off button 102 to protrude through it. The on/off button 102 provides a means for activating the camera 12. The location of the on/off button 102 can vary on the camera 12.

Referring to FIGS. 9-11, the second attachment portion 50′ also has another opening 104 which is sized and shaped to allow an on/off button 106 to protrude through it. The on/off button 106 provides a means for activating the monitor 90. The location of the on/off button 106 can vary on the camera 12.

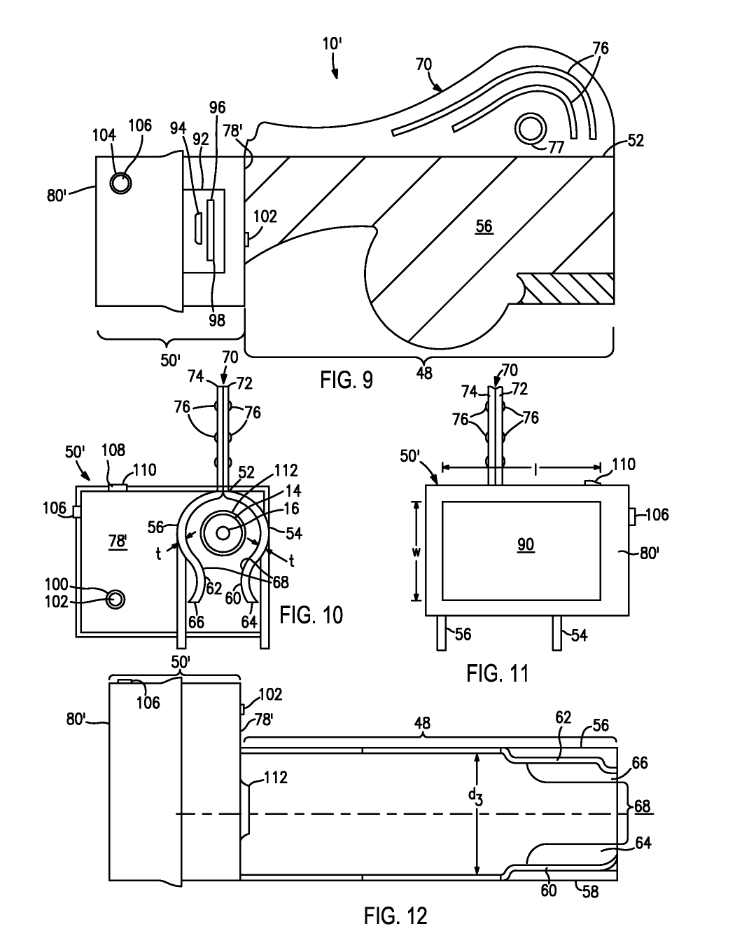

Referring to FIG. 10, the second attachment portion 50′ further has still another opening 108 which is sized and shaped to allow a picture taking button 110 to protrude through it. The picture taking button 110 provides a means for activating the shutter 14 on the camera 12 such that a photo, photograph or image can be taken through the lens 16. The location of the picture taking 110 can vary on the camera 12 but generally is located on an upper surface so that it can be easily depressed by a person's index finger while the person is holding the housing 10′ in his or her hand.

Referring to FIG. 12, the second attachment portion 50′ has an aperture 112 formed through the first surface 78′. The aperture 112 is aligned with the shutter 14 and the lens 16 of the camera 12. In addition, the aperture 112 is aligned with the aperture 26 formed through the tubular barrel 22 when the tubular barrel 22 is secured in the first attachment portion 48′ of the housing 10′. The housing 10′ functions in optically aligning the shutter 14 and the lens 16 of the camera 12 along the longitudinal central axis X-X of the tubular barrel 22 and the aperture 26 formed therein.

It should be understood that the second attachment portion 50′ can have additional openings for exposing addition ports located on the camera 12, if desired.

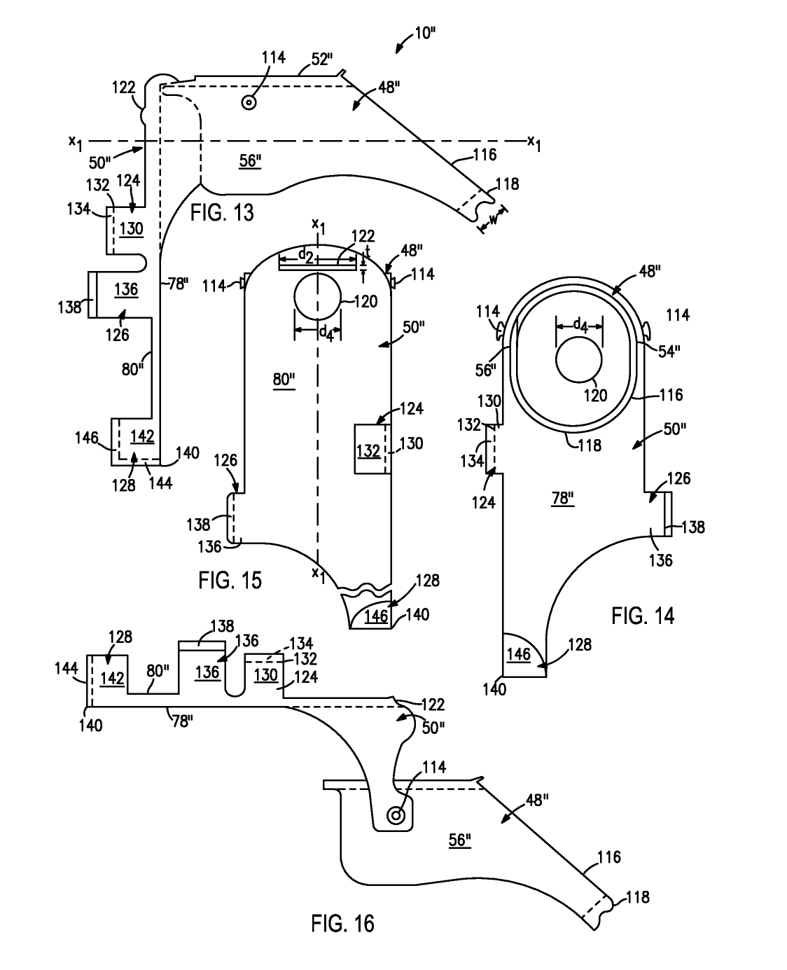

Referring now to FIGS. 13-16, a third embodiment of the housing 10″ is depicted. The housing 10″ functions to optically align and secure a camera 12 to an aperture 26 formed through the tubular barrel 22 of the scope 20. The housing 10″ has a first attachment portion 48″ and a second attachment portion 50″. In this embodiment, the first attachment portion 48″ is movably mounted to the second attachment portion 50″. By “movable mounted” it is meant that the first attachment portion 48″ can rotate, move or pivot relative to the second attachment portion 50″. The first attachment portion 48″ can be movably mounted to the second attachment portion 50″ by a variety of mechanisms. As illustrated, a pivot pin 114 is used to allow the first attachment portion 48″ to rotate or pivot relative to the second attachment portion 50″. However, other mechanical attachment mechanisms can be used which are known to those skilled in the art. Cams, gears, slides, wheels, brackets, etc. can all be used instead of, or in addition to, the pivot pin 114.

Referring to FIGS. 13 and 14, the first attachment portion 48″ also has a different shape in that a collar 116 is formed on its forward end. The collar 116 has a neck portion 118 which is relatively narrow in width w. The width w of the neck portion 118 can range from about 0.25 inches to about 0.75 inches. Desirably, the width w of the neck portion 118 is less than about 0.5 inches. More desirably, the width w of the neck portion 118 is about 0.4 inches or less. The collar 116 has an oval cross-sectional shape and encloses 360 degrees. The collar 116 is designed to fit over the first end 28 of the scope 20 such that the neck portion 118 rests in front of the handle 24. The collar 116 cooperates with a pair of sidewalls 54″ and 56″ to hold the housing 10″ secure to the tubular barrel 22 of the scope 20. The pair of sidewalls 54″ and 56″ is similar to and performs the same function as the pair of sidewalls 54 and 56, shown in FIGS. 1 and 5-8. One difference is that the pair of sidewalls 54″ and 56″ do not have feet 60 and 62. In other words, the pair of sidewalls 54′ and 56″ is shorter in overall length. The reason the feet 60 and 62 are not present is that the collar 116 provides a tight securement onto the tubular barrel 22 and prevents the first attachment portion 48″ from moving vertically relative to the tubular barrel 22. Therefore, the collar 116 takes the place of the neck portion 68 shown in FIG. 5. The housing 10″ also does not include the grasping mechanism 70 since the housing 10″ is designed to slide over the first end 28 and onto the tubular barrel 22 instead of being pressed down onto the upper surface 46 of the tubular barrel 22.

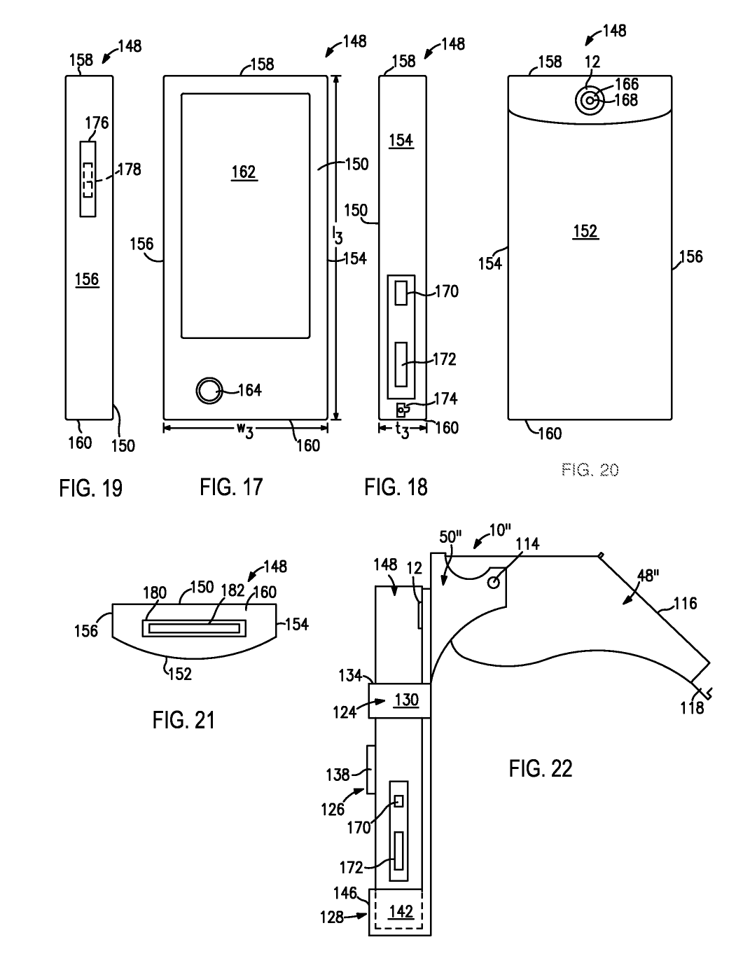

Referring to FIGS. 13 and 14, the second attachment portion 50″ of the housing 10″ is also different from the second attachment portions 50 or 50′ shown in the first two embodiments of the housing 10 or 10′. The second attachment portion 50″ has a longitudinal central axis X₁-X₁. The first attachment portion 50″ is designed to receive an external device 148, see FIG. 17-21, which is a camera 12 or which includes a camera 12. The external device 148 can be a camera 12, a cellular phone, a smart phone such as an iPhone, or some other electronic device which has a built-in camera 12. The housing 10″ is not designed to completely enclose the external device 148 but can permanently hold the external device 148 in optical alignment with the tubular barrel 22 of the scope 20. Primarily, the second attachment portion 50″ is structurally shaped to receive and temporarily retain a camera 12 or an external device 148, which includes a camera, in optical alignment with the tubular barrel 22 of the scope 20. The second attachment portion 50″ is especially adapted to allow a cellular phone or a smart phone to be inserted therein and to be later removed therefrom easily and quickly. The external device 148 can be inserted and then removed from the second attachment portion 50″ an infinite number of times.

Referring to FIGS. 13-16, the second attachment portion 50″ has a first surface 78″ and a second surface 80″. The first surface 78″ faces the first attachment portion 48″ while the second surface faces away from the first attachment portion 48″. Both the first and second surfaces 78″ and 80″ are approximately planar in configuration. An aperture 120 is formed through the second attachment portion 50″, see FIG. 15. The aperture 120 can vary in size and shape. Desirably, the aperture 120 is circular in shape and has a diameter d₄ of at least about 0.25 inches. More desirably, the aperture 120 has a diameter d₄ of at least about 0.5 inches. Even more desirably, the aperture 120 has a diameter d₄ of about 0.75 inches. The aperture 120 permits the shutter 14 and the lens 16 of the camera 12 or of the external device 148 which includes a camera, to be optically aligned which the aperture 26 formed through the tubular barrel 22 of the scope 20 which is secured to the first attachment portion 48″.

Referring to FIGS. 13, 14 and 16, the second attachment portion 50″ also includes a ridge 122 formed on the second surface 80″ and adjacent to the aperture 120. The ridge 122 has a length l₂ and a thickness t₂. The length l₂ is at least about 0.75 inches. Desirably, the length l₂ is about 1 inch. The thickness t₂ can range from between about 0.05 inches to about 0.2 inches. Desirably, the thickness t₂ is about 0.1 inches. The ridge 122 functions to hold the camera 12, or the external device 148 which includes a camera, perpendicular or at approximately 90 degrees to the second surface 80″ such that the lens 16 of the camera 12, or the external device 148 which includes a camera, can be properly aligned with the aperture 26 formed through the tubular barrel 22 of the scope 20 mounted in the first attachment portion 48″.

Still referring to FIGS. 13-16, the housing 10″ further includes a plurality of securement devices 124, 126 and 128. Three securement devices 124, 126 and 128 are shown but fewer or more can be utilized, as needed. Each of the securement devices 124, 126 and 128 can vary in size and configuration. The securement devices 124, 126 and 128 can be identical in construction or can differ in construction. Desirably, each of the securement devices 124, 126 and 128 differ in construction. All of the securement devices 124, 126 and 128 are situated or located on a periphery of the second attachment portion 50″ and extend inwardly from the periphery of the second attachment portion 50″.

Referring to FIG. 15, the first securement device 124, that being the one located closest to the aperture 120, is constructed as an L-shaped hook having a first leg 130 with a distal end 132 joined to a second leg 134. The first leg 130 extends perpendicularly outward from the periphery of the second surface 80″ and the second leg 134 extends perpendicularly inward from the periphery of the second surface 80″ towards the longitudinal central axis X₁-X₁ of the second attachment portion 50″. The second securement device 126 is situated vertically below and offset from the first securement device 124. The second securement device 126 has a first leg 136 joined to the opposite side of the second surface 80″. The first leg 136 extends perpendicularly outward from the periphery of the second surface 80″. The first leg 136 has a distal end 138 having a semi-circular profile. The distal end 138 extends inward and then outward from the longitudinal central axis X₁-X₁. The semi-circular distal end 138 does not extend inward towards the longitudinal central axis X₁-X₁ as much as the second leg 134 of the first securement device 124. As shown in FIG. 15, the second securement device 126 is offset from the first securement device 124. One can consider the second securement device 126 to have a modified L-shaped hook configuration.

Still referring to FIGS. 13-16, the third securement device 128 is positioned at a corner 140 of the second attachment portion 50″. As shown, the second attachment portion 50″ has a generally rectangular configuration with a pair of side edges, a curved top end and a curved lower end. The point of intersection between one of the side edges and the curved lower end forms a corner 140. The third securement device 128 has a first side 142, a second side 144 and a third side 146. The first side 142 extends perpendicularly outward from one of the side edges of the second surface 80″. Desirably, the third securement device 128 is located on the same side of the second surface 80″ as is the first securement device 124. The third securement device 128 is vertically spaced apart from both the first securement device 124 and the second securement device 126. The distance between the three securement devices 124, 126 and 128 can vary. Desirably, the distance between two adjacent securement devices 124, 126 and 128 will range from between about 0.5 inches to about 3 inches. More desirably, the distance between two adjacent securement devices 124, 126 and 128 will range from between about 0.75 inches to about 2 inches. It should be noted that the distance separating the first and second securement devices, 124 and 126 respectively, can be equal to or different from the distance separating the second and third securement devices, 126 and 128 respectively.

The second side 144 extends perpendicularly outward from the bottom end of the second surface 80″. The third side 146 is joined to both the first and second sides 142 and 144 and extends inward towards the longitudinal central axis X₁-X₁. The first, second and third sides, 142, 144 and 146 respectively, cooperate with the second surface 80″ to form a scoop or cup shaped member that can secure the camera 12 or the external device 148 which includes a built-in camera, in the X, Y and Z directions. This is important because the camera 12, or the external device 148 which includes a built-in camera, must be held secure and in optical alignment with the aperture 120 formed in the second attachment portion 50″. In such a position, the camera 12, or the external device 148 which includes a built-in camera, will be optically aligned with the aperture 26 formed through the tubular barrel 22 of the scope 20.

It should be understood that the securement device 126 can alternatively be located at the opposite lower corner of the second attachment portion 50″. Furthermore, the securement device 126 can have a scoop or cup shape similar to the construction of the securement device 128.

Referring now to FIGS. 17-22, the external device 148 having a built-in camera is depicted. The external device 148 can be a cellular phone, a smart phone, an iPhone, etc. The external device 148 can be easily and quickly inserted and then be removed from the housing 10″ or some other type of housing. The external device 148 does not have to be permanently secured to the housing 10″ but instead is removable from the housing 10″. The external device 148 can be inserted and then removed from the housing 10″ an infinite number of times. The external device 148 has a front surface 150, an oppositely aligned back surface 152, a right side 154, a left side 156, a top 158 and a bottom 160. The external device 148 has a generally rectangular profile with slightly rounded corners. The external device 148 has a length l₃, a width w₃ and a thickness t₃. The length l₃ can range from between about 3 inches to about 5 inches. Desirably, the length l₃ is about 4 inches. The width w₃ can range from between about 1 inch to about 3 inches. Desirably, the width w₃ is about 1.75 inches. The thickness t₃ can range from between about 0.25 inches to about 0.75 inches. Desirably, the thickness t₃ is about 0.5 inches. The front surface 150, best depicted in FIG. 17, is planar and includes a monitor 162. The monitor 162 can be formed from glass. The glass can be clear or tinted. The larger the overall size of the monitor 162 the better it is because it enables the words, text, numbers, icons, etc. that appear on the monitor 162 to be easier to read. The external device 148 contains internal electronics that run software applications that can perform various functions. Some typical applications loaded on cellular phones, smart phones, iPhones, etc. include but are not limited to: a Global Positioning System (GPS) which can determine geographical location, a calendar to identify the year, month and date, a clock to tell the current time in each of the 24 time zones, a calculator to allow the user to do mathematical calculations, various games which allow the user to play a number of different games, etc. The external device 148 also contains a certain amount of memory to record, store and display photos, photographs or images. The memory can also be used to save phone messages, display saved voice and/or text messages, etc.

The monitor 162 is shown occupying at least about 50% of the surface area of the front surface 150. Desirably, the monitor 162 occupies at least about 60% of the surface area of the front surface 150. Even more desirably, the monitor 162 occupies at least about 70% of the surface area of the front surface 150. Besides the monitor 162, the front surface 150 also has an activation mechanism 164 for turning the external device 148 on and off. The activation mechanism 164 can vary in construction and operation. For example, the activation mechanism 164 can be a button that can be depressed in order to turn the monitor 162 on and can then be depressed a second time to turn the monitor 162 off. The size and shape of the activation mechanism 164 can vary. Desirably, the activation mechanism 164 is a circular button having a diameter of less than about 0.5 inches. More desirably, the activation mechanism 164 is a circular button having a diameter of about 0.37 inches.

Referring to FIGS. 20 and 21, the back surface 152 of the external device 148 is shown having a curved or arcuate surface to allow the user to hold the external device 148 easily in the palm of his or her hand. A camera 12 is mounted in the external device 148 and has a shutter 166 and a lens 168. The shutter 166 is capable of opening and closing around the lens 168. When the shutter 166 is completely dosed, the lens 168 is hidden. When the shutter 166 is open, a picture can be taken through the lens 168 of the camera 12. The shutter 166 can partially open depending on the amount of light needed to take a photo or picture.

Referring to FIG. 18, the right side 154 of the external device 148 includes an on/off button 170 and an activation mechanism 172 for working the camera 12. The activation mechanism 172 can be a circular button similar in function as the activation mechanism 164. An icon 174 is formed in the right side 154 adjacent to the camera activation mechanism 172 so as to alert the user that this button is used to take photos or pictures. The icon is an optional but a handy feature.

Referring to FIG. 19, the left side 156 of the external device 148 contains a removable panel 176 which covers a Universal Serial Bus (USB) port 178. The removable panel 176 can be tethered by a connecting strap or wire (not shown) which prevents the panel 176 from being completely separated from the external device 148. The USB port 178 allows information, including photos, photographs, pictures, images, etc. which have been recorded by the camera 12 to be electronically transferred, via an electrical cable, to another electronic device for storage and/or viewing. The other electronic device can be a computer, a monitor, a television set, an iPad, etc.

Referring again to FIG. 21, the bottom 160 of the external device 148 contains a port 180 which houses a removable memory card 182. The memory card 182 can be removed and be directly inserted into some other electronic device. The other electronic device can be a photo reader, a photo printer, a computer, a monitor, a television set, an iPad, etc.

Referring to FIG. 22, the external device 148 is shown being securely retained in the second attachment portion 50″ of the housing 10″. The external device 148 is inserted downward into the top of the second attachment portion 50″ such that the back surface 152 faces toward the first attachment portion 48″. The bottom 160 of the external device 148 will contact the securement device 128 while the right and left sides, 154 and 156 respectively, will be held secure by the securement devices, 124 and 126 respectively. The ridge 122 will center the external device 148 so that the camera 12, with its shutter 166 and lens 168, is aligned perpendicular to the aperture 120 formed in the second attachment portion 50″. In this position, the camera 12 will also be optically aligned with the aperture 26 formed through a tubular barrel 22 of a scope 20 and when the camera 12 is activated, the external device 148 can capture and record an image of what is seen through the scope 20.

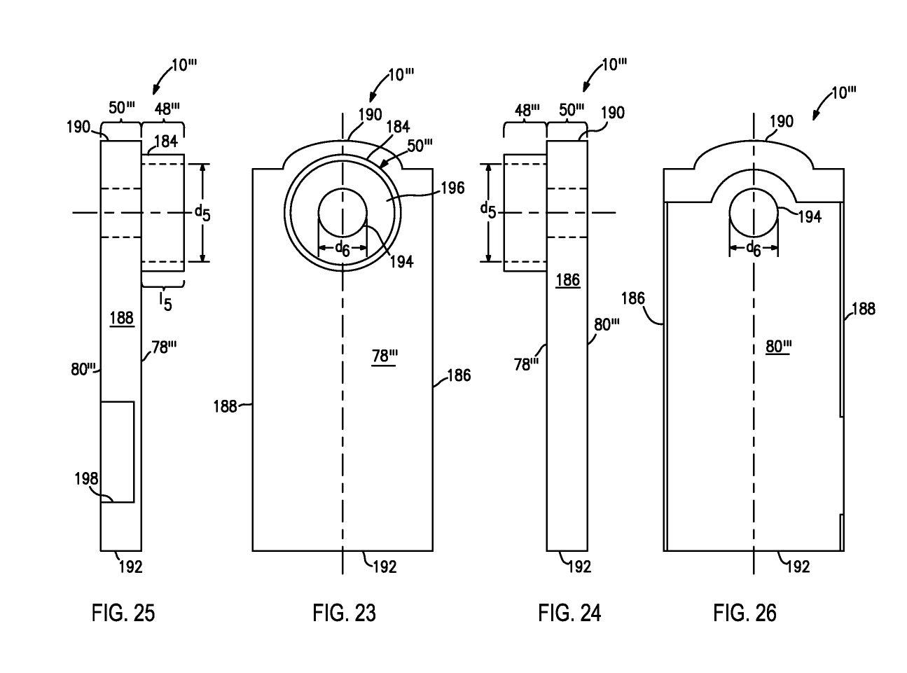

Referring now to FIGS. 23-26 a fourth embodiment of a housing 10′″ is depicted. The housing 10′″ has a first attachment portion 48′″ secured to a second attachment portion 50′″. The first attachment portion 48′″ is formed in the configuration of a collar 184. The collar 184 is a hollow, circular ring designed to encircle the outside diameter d of the tubular barrel 22 of the scope 20. The collar 184 has an internal diameter d₅, a length l₅, a ring thickness t₅. The internal diameter d₅ is sized to snuggly fit over at least a portion of the outside diameter d of the tubular barrel 22. The internal diameter d₅ of the collar 184 can form a frictional fit with the outside diameter d of the tubular barrel 22 of the scope 20. Alternatively, the internal diameter ds of the collar 184 can form a snug fit with the outside diameter d of the tubular barrel 22 of the scope 20. Desirably, the fit between the internal diameter d₅ of the collar 184 and the outside diameter d of the tubular barrel 22 will be sufficiently strong to hold the housing 10′″ secure to the tubular barrel 22 of the scope 20. The collar 184 is removably secured to the tubular barrel 22 of the scope 20 which permits it to be positioned on and then later be removed from the tubular barrel 22 of the scope 20. It is foreseen that the collar 184 with be secured to and then be removed from the tubular barrel 22 of the scope 20 hundreds and maybe thousands of times over the life of the housing 10′″.

The length l₅ of the collar 184 cooperates with the internal diameter d₅ of the collar 184 to obtain a secure fit onto the tubular barrel 22 of the scope 20. The length l₅ of the collar 184 can range from between about 0.1 inches to about 5 inches. Desirably, the length l₅ of the collar 184 can range from between about 0.25 inches is at least about 2 inches. More desirably, the length l₅ of the collar 184 is at least about 0.5 inches. Even more desirably, the length l₅ of the collar 184 can range from between about 0.25 inches to about 1 inch. The thickness t₅ of the ring portion of the collar 184 also factors into the strength of the collar 184 and its ability to form a secure fit onto the tubular barrel 22 of the scope 20. The thickness t₅ of the ring portion of the collar 184 can range from between about 0.01 inches to about 0.5 inches. Desirably, the thickness t₅ of the ring portion of the collar 184 can range from between about 0.1 inches to about 0.4 inches. More desirably, the thickness t₅ of the ring portion of the collar 184 can range from between about 0.15 inches to about 0.3 inches.

The second attachment portion 50′″ has a first surface 78′″, a second surface 80′″ and an aperture 194 formed therethrough. The second surface 80′″ is oppositely aligned to the first surface 78′″. The second attachment portion 50′″ also has a right side 186, a left side 188, a top 190 and a bottom 192. The aperture 194 can vary in size and geometrical shape. Desirably, the aperture 194 is circular and has a diameter d₆. The diameter d₆ can range from between about 0.25 inches to about 1 inch. Desirably, the diameter d₆ can range from between about 0.3 inches to about 0.75 inches. More desirably, the diameter de can range from between about 0.3 inches to about 0.6 inches. The aperture 194 provides an opening such that the shutter 166 and lens 168 of the external device 148, having a built-in camera, which is secured to the second attachment portion 50′″ can be optically aligned with the aperture 26 formed in the tubular barrel 22 of the scope 20.

Referring again to FIG. 23, one can clearly see that a portion of the first surface 78′″ can act as a back wall 196 of the collar 184 to prevent the tubular barrel 22 of the scope 20 from contacting the external device 148 and its shutter 166 and lens 168. The back wall 196 will act as a stop to limit the amount the collar 184 can encircle the outside diameter d of the tubular barrel 22.

Referring to FIG. 25, one can see that the left side 188 of the second attachment portion 50′″ contains an opening 198. The opening 198 can vary in size and geometrical shape. The opening 198 is depicted as a rectangular opening which will align with the on/off button 170 and the activation mechanism 172 for the camera of the external device 148, see FIG. 18.

It should be understood that if the on/off button 170 and/or the activation mechanism 172 is located in a different position on the external device 148, then the opening 198 can be moved to the right side 186 of the second attachment portion 50′″ or somewhere else on the left side 188 to accommodate such a location.

The invention has been described above as relating to the housings 10, 10′, 10″ and 10′″. However, this invention also relates to the combination of a camera 12 or an external device 148 which includes a built-in camera, and a housing 10, 10′, 10″ or 10′″. The housing 10, 10′, 10″ or 10′″ has a first attachment portion 48, 48′, 48″ or 48′″ that is sized and configured to be removeably secured to at least one end of the tubular barrel 22 of the scope 20. The tubular member 22 has a longitudinal central axis X-X and an aperture 26 formed therethrough. The aperture 26 is coaxially aligned with the longitudinal central axis X-X. The housing 10, 10′, 10″ or 10′″ has a second attachment portion 50, 50′, 50″ or 50′″ which includes a first surface 78, 78′, 78″ or 78′″ secured to said first attachment portion 48, 48′, 48″ or 48′″. The housing 10, 10′, 10″ or 10′″ also has a second surface 80, 80′, 80″ or 80′″ sized and configured to have the camera 12 or the external device 148 which includes a built-in camera, removably secured thereto. The second surface 80, 80′, 80″ or 80′″secures the camera 12 or the external device 148 which includes a built-in camera, to the tubular barrel 22 of the scope 20 and in optical alignment with the aperture 26 formed through the tubular barrel 22.

In the combination of the camera 12 or the external device 148 which includes a built-in camera, the housing 10, 10′, 10″ or 10′″, and the scope 20 can be an opthalmoscope. In this combination, the housing 10, 10′, 10″ or 10′″ has a first attachment portion 48, 48′, 48″ or 48′″ sized and configured to be removeably secured to at least one end of the tubular member 22 of the opthalmoscope. The housing 10, 10′, 10″ or 10′″ also has a second attachment portion 50, 50′, 50″ or 50′″ with a first surface 78, 78′ 78″ or 78′″ secured to the first attachment portion 48, 48′, 48″ or 48′″ and a second surface 80, 80′, 80″ or 80′″ sized and configured to have the camera 12 or the external device 148 which includes a built-in camera, removably secured thereto. The camera 12 or the external device 148 which includes a built-in camera, is then held secured to the opthalmoscope 20 and in optical alignment with the aperture 26 formed through the tubular member 22.

In this combination, the opthalmoscope 20 can be a hand held, portable device. The opthalmoscope 20 can have a power pack formed in its handle 24. The first attachment portion 78′″ can be a hollow circular collar 184 sized to snuggly fit over at least a portion of the outside diameter d of the tubular member 22, and the collar 184 can have a length l₅ of at least about 0.5 inches.

### Method

This invention also includes a method for optically aligning and securing a camera 12 or the external device 148 which includes a built-in camera, to a tubular barrel 22 of a scope 20 using a housing 10, 10′, 10″ or 10′″. The tubular member 22 has a longitudinal central axis X-X and an aperture 26 formed therethrough. The housing 10, 10′, 10″ or 10′″ has a first attachment portion 48, 48′, 48″ or 48′″, a second attachment portion 50, 50′, 50″ or 50′″, and an aperture 26, 112, 120 or 194 formed therethrough. The first attachment portion 48, 48′, 48″ or 48′″ is sized and configured to be removeably secured to at least one end of the tubular member 22. The second attachment portion 50, 50′, 50″ or 50′″ has a first surface 78, 78′, 78″ or 78′″ secured to the first attachment portion 48, 48′, 48″ or 48′″ and a second surface 80, 80′, 80″ or 80′″ sized and configured to have a camera 12 or the external device 148 which includes a built-in camera, removably secured thereto. The method includes the steps of positioning and securing the first attachment portion 48, 48′, 48″ or 48′″ onto one end of the tubular member 22 of a scope 20 and then inserting the camera 12 or the external device 148 which includes a built-in camera, into the second attachment portion 50, 50′, 50″ or 50′″ such that the camera 12 or the external device 148 which includes a built-in camera, is in optical alignment with both the aperture 26, 112, 120 or 194 formed through the second attachment portion 50, 50′, 50″ or 50′″ and the aperture 26 formed through the tubular member 22 of the scope 20.

The method can further include positioning an opposite end of the scope 20 adjacent to a patient's eye and activating the camera 12 to take a photo, picture or image of the patient's eye.

The method can also include electronically sending the photo, picture or image to a monitor and viewing the photo, picture or image on the monitor.

The method can still further include electronically storing the various photos, pictures or images on a computer and viewing the photos, pictures or images at a later time.

This invention further includes a method for optically aligning and securing a camera 12 or the external device 148 which includes a built-in camera, to the tubular barrel 22 of the scope 20 using a housing 10, 10′, 10″ or 10′″. The camera 12 or the external device 148 which includes a built-in camera, has an activation means 172 for taking photos, pictures or images and a monitor 162 for displaying the photos, pictures or images. The tubular member 22 has a longitudinal central axis X-X and an aperture 26 formed therethrough. The housing 10, 10′, 10″ or 10′″ has a first attachment portion 48, 48′, 48″ or 48′″, a second attachment portion 50, 50′, 50″ or 50′″, and an aperture 26, 112, 120 or 194 formed therethrough. The first attachment portion 48, 48′, 48″ or 48′″ is sized and configured to be removeably secured to at least one end of the tubular member 22. The second attachment portion 50, 50′, 50″ or 50′″ has a first surface 78, 78′, 78″ or 78′″ secured to the first attachment portion 48, 48′, 48″ or 48′″ and a second surface 80, 80′, 80″ or 80′″ sized and configured to have the camera 12 or the external device 148 which includes a built-in camera, secured thereto. This method includes the steps of positioning the camera 12 or the external device 148 which includes a built-in camera, in the second attachment portion 50, 50′, 50″ or 50′″ and then securing the first attachment portion 48, 48′, 48″ or 48′″ onto one end of the tubular member 22 such that the camera 12 or the external device 148 which includes a built-in camera, is in optical alignment with both the aperture 26, 112, 120 or 194 formed through the second attachment portion 50, 50′, 50″ or 50′″ and the aperture 26 formed through the tubular member 22.

### Kit

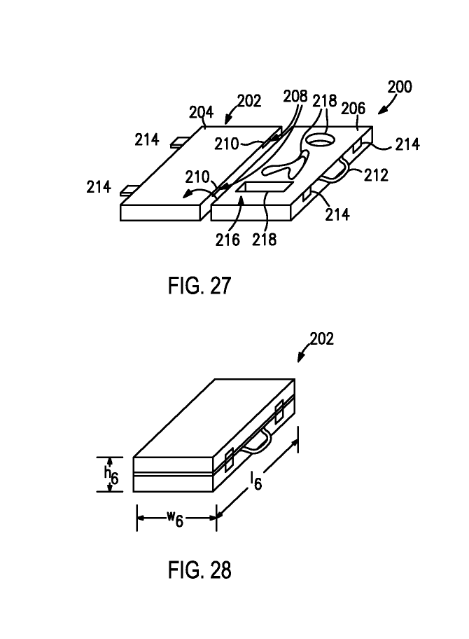

Referring now to FIGS. 27 and 28, this invention also covers a kit 200 which includes a case 202. The case 202 can vary in dimensions. The case 202 has a length l₆, a width w₆ and a height h₆. The case 202 can have a length l₆ which ranges from between about 10 inches to about 36 inches, a width w₆ which ranges from between about 8 inches to about 24 inches, and a height h₆ which ranges from between about 4 inches to about 8 inches. Desirably, the case 202 can have a length l₆ which ranges from between about 10 inches to about 24 inches, a width w₆ which ranges from between about 8 inches to about 18 inches, and a height h₆ which ranges from between about 4 inches to about 6 inches.

The case 202 is depicted in an open position in FIG. 27 and in a closed position in FIG. 28. In the open position, the contents within the case 202 are revealed. In the closed position, the case 202 is closed and can be carried about by a person. The case 202 has a first portion 204 joined to a second portion 206. The first and second portions, 204 and 206 respectively, are joined to one another by an attachment mechanism 208. The attachment mechanism 208 can vary. For example, the attachment mechanism 208 can be one or more hinges. In FIG. 27, a pair of spaced apart hinges 210, 210 are depicted. Each hinge 210 can be mechanical hinge or a living hinge. A living hinge 210 is a hinge that is constructed from a moldable material that can be opened and closed multiple times without breaking. Some plastic and thermoplastic materials have the characteristics that qualify them to be used as living hinges. The attachment mechanism 208 can also include various other kinds and types of mechanical devices that can function to permit the first portion 204 to pivot or rotate relative to the second portion 206. The attachment mechanism 208 is designed to allow the first portion 204 of the case to open and close relative to the second portion 206 of the case 202. The first portion 204 could be constructed to rotate, pivot or move relative to the second portion 206. In the dosed position, see FIG. 28, the first and second portions, 204 and 206 respectively, will be aligned adjacent to one another to form a closed or enclosed case 202. In the open position, see FIG. 27, the first portion 204 will rotate or pivot away from the second portion 206 by an angle of about 180 degrees or more. In FIG. 27, the first portion 204 is rotated 180 degrees relative to the second portion 206.

The case 202 can also include a handle 212. The handle 212 can vary in size and configuration. The handle 212 is shown being secured to the second portion 206 of the case 202 although it could be secured to the first portion 204 of the case 202, if desired. The case 202 can also contain one or more locking mechanisms 214 which function to lock or secure the first and second portions, 204 and 206 respectively, together. Two spaced apart locking mechanisms 214, 214 are shown in FIG. 27. A first portion of each locking mechanism 214 can be secured to the first portion 204 of the case 202 and a second portion of each of the locking mechanisms 214, 214 can be secured to the second portion 206 of the case 202. The locking mechanism 214 is capable of securing the first portion 204 of the case 202 to the second portion 206 of the case 202 when the first and second portions, 204 and 206 respectively, are in their closed position. Various types of locking mechanisms 214 can be used.

The case 202 also includes at least one foam member 216 which is sized to snugly fit or be positioned or retained in one of the first or second portions, 204 or 206 of the case 202. For example, the foam member 216 can be frictionally held in one of the first or second portions, 204 or 206 respectively, of the case 202. In FIG. 27, the foam member 216 is shown being sized and form fitted into the second portion 206 of the case 202. The foam member 216 could be held firm in both of the first or second portions, 204 or 206 respectively, of the case 202, if desired. There are many ways to secure the foam member 216 into one of the first and/or second portions, 204 and/or 206 respectively, of the case 202. For example, the foam member 216 can be glued in place. Alternatively, the foam member 216 can be held in place by a mechanical fastener. Still another way is to size the foam member 216 to frictional fit into one of the first and second portions, 204 and 206 respectively. Those skilled in the art will know of many other ways to fit the foam member 216 into one or both portions, 204 and 206 respectively, of the case 202.

The foam member 216 can have one or more separate and distinct cutouts 218. In FIG. 3, three cutouts 218, 218 and 218 are depicted for discussion purposes. However, in actual practice, multiple cutouts 218 can be formed in the foam member 216. Each of the cutouts 218 can be sized and shaped to accommodate a particular object or device. Each cutout 218 is a 3-dimensional opening having a length, a width and a depth. The geometrical configuration of any particular cutout 218 can vary. Each cutout 218 can be sized and configured to frictionally engage a unique object or device that is intended to be temporally stored and/or secured therein. For example, a portable, hand held opthalmoscope 20 can be inserted into the approximately T-shaped cutout 218 shown in FIG. 27. The opthalmoscope 20 will be held secure in the cutout 218 by the moldable characteristics of the foam member 216. The ability of the foam member 216 to contract or deform as required about the periphery of the opthalmoscope 20 means that the cutout 218 does not have to be configured to the exact dimensions of the opthalmoscope 20. The foam member 216 can temporally form to the shape of the opthalmoscope 20. The opthalmoscope 20 will be removably positioned in the cutout 218 and can be easily and quickly removed from the cutout 218 when needed. The opthalmoscope 20 is depicted as having a tubular barrel 22 with an aperture 26 formed therethrough. However, this invention can utilize various types of scopes 20 and some may have drastically different shapes.

A camera 12 or an external device 148 having a built-in camera, can also be removably positioned in one of the cutouts 218. The camera 12 or an external device 148 having a built-in camera, will have an activation mechanism 172 which can be pressed or somehow moved so as to take a photo, picture or image. The housing 10, 10′, 10″ or 10′″ can also be positioned in one of the cutouts 218. The housing 10, 10′, 10″ or 10′″ is capable of optically aligning and securing the camera 12 or an external device 148 having a built-in camera, to the aperture 26 formed through the tubular barrel 22 of an opthalmoscope 20. The housing 10, 10′, 10″ or 10′″ includes a first attachment portion 48, 48′, 48″ or 48′″, and a second attachment portion 50, 50′, 50″ or 50′″, and an aperture 26, 112, 120 or 194 formed through. The first attachment portion 48, 48′, 48″ or 48′″ is sized and configured to be removeably secured to at least one end of the opthalmoscope 20. The second attachment portion 50, 50′, 50″ or 50′″ has a first surface 78, 78′, 78″ or 78′″ secured to the first attachment portion 48, 48′, 48″ or 48′″ and a second surface 80, 80′, 80″ or 80′″ sized and configured to have the camera 12 or an external device 148 having a built-in camera, removably secured thereto. In this arrangement, the camera 12 is in optical alignment with both the aperture 26, 112, 120 or 194 formed through the second attachment portion 50, 50′, 50″ or 50′″ and the aperture 26 formed through the tubular member 22 of the opthalmoscope 20.

Various other objects or devices can also be retained in one of the cutouts 218. Such objects or devices include but are not limited to: a computer with a built-in monitor, an electrical cable, a USB cable for connecting the camera 12 to a computer, a computer mouse which can be connected to the computer and which can function to manipulate the computer to control images displayed on the monitor, a power supply for operating the opthalmoscope 20, a set of tools for making adjustments to the opthalmoscope 20, a Universal Serial Bus (USB) flash drive for electronically storing images recorded by the camera 12, a power cord for operating the opthalmoscope 20, various adapters, etc. Almost any known object or device can be added to the kit 200 as required. An instruction manual can also be included in the kit 200.

While the invention has been described in conjunction with several specific embodiments, it is to be understood that many alternatives, modifications and variations will be apparent to those skilled in the art in light of the foregoing description. Accordingly, this invention is intended to embrace all such alternatives, modifications and variations which fall within the spirit and scope of the appended claims.

I claim:

1. A housing for optically aligning and securing a camera to an aperture formed through a tubular barrel of a scope, comprising:
a) a first attachment portion sized and configured to be removeably secured to at least a portion of said tubular barrel of said scope; and
b) a second attachment portion having a first surface, a second surface and an aperture formed therethrough, said first surface being secured to said first attachment portion, said second surface sized and configured to removably retain a camera, such that said camera is in optical alignment with both said aperture formed through said second attachment portion and said aperture formed through said tubular barrel of said scope.

2. The housing of claim 1 wherein said tubular barrel has an outer surface and a longitudinal central axis, and said aperture formed through said tubular barrel is coaxially aligned with said longitudinal central axis, said outer surface of said tubular barrel has a coefficient of friction, and said first attachment portion is formed from a material having a coefficient of friction which is different from said coefficient of friction of said outer surface of said tubular barrel.

3. The housing of claim 2 wherein said first attachment portion is a hollow circular collar sized to snuggly fit over at least a portion of said outer surface of said tubular barrel, said collar having a length ranging from between about 0.1 inches to about 5 inches, and a thickness ranging from between about 0.01 inches to about 0.5 inches.

4. The housing of claim 1 wherein said first attachment portion is a C-shaped member spanning an arc of at least about 270 degrees and having an opening, said C-shaped member having an internal diameter which is slightly larger than said outer surface of said tubular barrel, and said second attachment portion is configured such that at least about 75% of a major surface of said camera is exposed.

5. The housing of claim 4 wherein said first attachment portion has a top wall and a pair of flexible sidewalls extending outward and downward from said top surface to form said C-shaped member having an open bottom, a pair of feet each being joined to one of said pair of flexible sidewalls, and a dimension located between said pair of feet which creates a narrow neck portion.

6. The housing of claim 5 wherein each of said pair of flexible sidewalls has a resiliency to return to its initial position once a force required to make each of them flex outward has been removed.

7. The housing of claim 1 wherein said first attachment portion is pivotably mounted to said second attachment portion and can rotate through an angle of at least about 90 degrees.

8. The housing of claim 1 wherein said second attachment portion has a periphery with at least three securement devices formed thereon, said securement devices cooperating to secure a camera to said housing, and at least one of said securement devices having an L-shaped profile with a first leg joined to said periphery of said second attachment portion and a second leg joined to said first leg, said first leg extending outward from said second surface and said second leg extending inward from said periphery.

9. The housing of claim 1 wherein said second attachment portion has a generally rectangular configuration which is sized and configured to temporarily receive a cellular phone having a camera built therein.

10. A combination comprising:
a) a camera; and
b) a housing having a first attachment portion sized and configured to be removeably secured to at least one end of a tubular barrel of a scope, said tubular barrel having an aperture formed therethrough, and a second attachment portion having a first surface, a second surface and an aperture formed therethrough, said first surface being secured to said first attachment portion, said second surface sized and configured to removably retain said camera, such that said camera is in optical alignment with both said aperture formed through said second attachment portion and said aperture formed through said tubular barrel of said scope.

11. The combination of claim 10 wherein said scope is an opthalmoscope, said tubular barrel has an outside diameter, and said first attachment portion is a hollow circular collar sized to snuggly fit over at least a portion of said outside diameter of said tubular barrel, and said collar having a length of at least about 0.25 inches.

12. A combination comprising:
a) an opthalmoscope having a tubular barrel with an aperture formed therethrough, said tubular barrel having an outside diameter; and
b) a housing having a first attachment portion sized and configured to be removeably secured to at least one end of said tubular barrel, and a second attachment portion having a first surface, a second surface and an aperture formed therethrough, said first surface being secured to said first attachment portion and said second surface sized and configured to removably retain a camera, such that said camera is in optical alignment with both said aperture formed through said second attachment portion and said aperture formed through said tubular barrel.

13. The combination of claim 12 wherein said opthalmoscope is a hand held portable device with a power pack, and said first attachment portion is a hollow circular collar sized to snuggly fit over at least a portion of said outside diameter of said tubular member, and said collar having a length of at least about 0.5 inches.

14. A method for optically aligning and securing a camera to a tubular barrel of a scope, said tubular barrel having an aperture formed therethrough, said method including the steps of:
a) forming a housing having a first attachment portion sized and configured to be removeably secured to at least one end of said tubular barrel, and a second attachment portion having a first surface, a second surface, and an aperture formed therethrough, said first surface being secured to said first attachment portion and said second surface sized and configured to removably retain said camera;
b) positioning and securing said first attachment portion onto one end of said tubular barrel; and
c) inserting said camera into said second attachment portion such that said camera is in optical alignment with both said aperture formed through said second attachment portion and said aperture formed through said tubular barrel.

15. The method of claim 14 further including positioning an opposite end of said scope adjacent to a patient's eye and activating said camera to take a picture of said patient's eye.

16. The method of claim 15 further including electronically sending said picture to a monitor and viewing said picture on said monitor.

17. The method of claim 16 further including electronically storing said picture on a computer and viewing said picture at a later time.

18. A method for optically aligning and securing a camera to a tubular barrel of a scope, said tubular barrel having an aperture formed therethrough, said camera having an activation mechanism for take pictures and a monitor for displaying said pictures, said method including the steps of:
a) forming a housing having a first attachment portion sized and configured to be removeably secured to at least one end of said tubular barrel, and a second attachment portion having a first surface, a second surface, and an aperture formed therethrough, said first surface being secured to said first attachment portion and said second surface sized and configured to removably retain said camera;
b) positioning and securing said first attachment portion onto one end of said tubular barrel;
c) inserting said camera into said second attachment portion such that said camera is in optical alignment with both said aperture formed through said second attachment portion and said aperture formed through said tubular barrel; and
d) activating said camera to take a picture and viewing said picture on said monitor.

19. A kit comprising:
a) a case having a first portion joined to a second portion, said first and second portions being movable between an open position wherein said case is open and a closed position wherein said case is closed;
b) a locking mechanism capable of securing said first portion to said second portion when said first and second portions are in said closed position;
c) at least one foam member positioned within at least one of said first and second portions, said foam member having at least three separate and distinct cutouts formed therein, each cutout being sized and configured to snuggly engage and temporarily hold an object inserted therein;
d) an opthalmoscope removably positioned in one of said cutouts, said opthalmoscope having a tubular barrel with an aperture formed therethrough;
e) a camera removably positioned in one of said cutouts, said camera having a button which can be activated to take a picture; and
f) a housing removably positioned in one of said cutouts, said housing capable of optically aligning and securing said camera to said aperture formed through said opthalmoscope, said housing having a first attachment portion sized and configured to be removeably secured to at least one end of said opthalmoscope, and a second attachment portion having a first surface, a second surface, and an aperture formed therethrough, said first surface being secured to said first attachment portion and a second surface sized and configured to retain said camera, such that said camera is in optical alignment with both said aperture formed through said second attachment portion and said aperture formed through said tubular barrel.

20. The kit of claim 19 further including a computer with a monitor, a cable for attaching said camera to said computer such that images recorded by said camera can be visually displayed on said monitor, a mouse electronically connected to said computer which can be manipulated to control images displayed on said monitor, a power supply for operating said opthalmoscope, a set of tools capable of being used to make adjustments to said opthalmoscope, a Universal Serial Bus (USB) flash drive for electronically storing images recorded by said camera, and a power cord for operating said opthalmoscope.
